(19) United States
(12) Patent Application Publication
(10) Pub. No.: US 2002/0042438 A1
(43) Pub. Date: Apr. 11, 2002

(54) Use of ergothioneine and/or its derivatives as an anti-glycation agent

(75) Inventors: Pascale Pelletier, Antony (FR); Herve Pageon, Puteaux (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 09/888,525

(22) Filed: Jun. 26, 2001

(30) Foreign Application Priority Data
Jun. 26, 2000 (FR) 0008158

(51) Int. Cl. A61K 31/4164

(52) U.S. Cl. 514/386

(57) ABSTRACT

The present invention includes a method for reducing or inhibiting the glycation of skin proteins, which includes topically applying a composition containing at least one selected from the group including ergothioneine, derivatives thereof, and mixtures thereof to the skin of a person in need thereof. Other embodiments of the invention include a method for making a composition for reducing the glycation of skin proteins, a method for reducing the glycation of collagen, a method for preventing or treating the signs of glycation-related ageing of the skin, a method for preventing or treating age-related loss of tonicity of the skin, a method for slimming or refining the silhouette or contours of the face, and a method for preventing or treating the orange-peel appearance of the skin.

### BACKGROUND OF THE INVENTION

1. Field of the Invention

The invention relates to the use of ergothioneine and/or its derivatives in a cosmetic composition, as an agent for reducing the glycation of skin proteins, in particular for preventing or treating the signs of ageing of the skin and/or the orange-peel appearance of the skin and for slimming and/or refining the silhouette and/or the contours of the face.

2. Discussion of the Background

Glycation is a non-enzymatic process involving a saccharide (glucose or ribose) which reacts according to the Maillard reaction with an amino group of an amino acid residue (such as, for example, lysine), particularly an amino acid residue of a protein, to form a Schiff s base. This Schiff s base, after undergoing an Amadori molecular rearrangement, can lead, via a sequence of reactions, to a bridging, particularly an intramolecular bridging such as, for example, of pentosidine type.

The above phenomenon is characterized by the appearance of glycation products whose content increases uniformly as a function of age. The glycation products are, for example, pyrraline, carboxymethyllysine, pentosidine, crossline, N^{ε}-(2-carboxyethyl)-lysine (CEL), glyoxallysine dimer (GOLD), methylglyoxallysine dimer (MOLD), 3DG-ARG imidazolone, versperlysines A, B and C, threosidine or the end products of advanced glycosylation (or AGEs).

The glycation of proteins is thus a universal phenomenon, which is well known in the skin, particularly in its dermal component, and mainly in collagen fibres. Specifically, the glycation of collagen increases uniformly with age, resulting in a uniform increase in the content of glycation products in the skin.

Without wishing to introduce any theory of skin ageing, it should be noted that other changes to collagen which might also be a consequence of glycation, for example, decrease in heat denaturation, increase in resistance to enzymatic digestion, and increase in intermolecular bridges, have been able to be demonstrated in the course of skin ageing (Tanaka S. et al., 1988, J. Mol. Biol., 203, 495-505; Takahashi M. et al., 1995, Analytical Biochemistry, 232, 158-162). Furthermore, changes due to the glycation of certain constituents of the basal membrane, for instance collagen IV, laminin and fibronectin, have been able to be demonstrated (Tarsio J F. et al., 1985, Diabetes, 34, 477-484; Tarsio J F. et al., 1988, Diabetes, 37, 532-539; Stemberg M. et al., 1995, C. R. Soc. Biol., 189, 967985). The entire contents of each of the above references are hereby incorporated by reference.

Thus, it is understood that, in the course of ageing of the skin, the physicochemical properties of the collagen change, and this collagen becomes more difficult to dissolve and to degrade. The result is a rigidification of the tissues, essentially leading to a loss of tonicity of the skin.

In addition to its effects on ageing of the skin, glycation is involved in the characteristic “orange-peel” appearance of cellulite. Specifically, in cellulite, the glycation of the collagen constituting the majority of the connecting sections results in a rigidification of the tissues, which then imprison the fat globules. The skin thus shows a succession of bumps formed by fatty lumps and of hollows formed by rigidified connecting sections, which are characteristic of the “orange-peel” appearance.

The importance of having available products which reduce or even inhibit the phenomenon of glycation of proteins may thus be appreciated.

Various products capable of inhibiting this glycation reaction are known: aminoguanidine (which is the inhibitor most commonly known), taurine, certain vitamins (B1, 136) and thiazolium derivatives.

### SUMMARY OF THE INVENTION

The present inventors have now discovered, surprisingly and unexpectedly, that ergothioneine and its derivatives have the property of reducing or even inhibiting the phenomenon of glycation of proteins and thus of acting firstly on the age-related loss of tonicity of the skin and secondly on the “orange-peel” appearance of the skin.

Accordingly, one embodiment of the present invention includes a method for reducing or inhibiting the glycation of skin proteins, which includes topically applying a composition containing at least one selected from the group including ergothioneine, derivatives thereof, and mixtures thereof to the skin of a person in need thereof.

Another embodiment of the present invention includes a method for making a composition for reducing the glycation of skin proteins, which includes contacting at least one selected from the group including ergothioneine, derivatives thereof, and mixtures thereof with a physiologically acceptable medium.

Another embodiment of the present invention includes a method for reducing the glycation of collagen, which includes topically applying a composition containing at least one selected from the group including ergothioneine, derivatives thereof, and mixtures thereof to the skin of a person in need thereof.

Another embodiment of the present invention includes a method for preventing or treating the signs of glycation-related ageing of the skin, which includes topically applying a composition containing at least one selected from the group including ergothioneine, derivatives thereof, and mixtures thereof to the skin of a person in need thereof.

Another embodiment of the present invention includes a method for preventing or treating age-related loss of tonicity of the skin, which includes topically applying a composition containing at least one selected from the group including ergothioneine, derivatives thereof, and mixtures thereof to the skin of a person in need thereof.

Another embodiment of the present invention includes a method for slimming or refining the silhouette or contours of the face, which includes topically applying a composition containing at least one selected from the group including ergothioneine, derivatives thereof, and mixtures thereof to the skin of a person in need thereof.

Another embodiment of the present invention includes a method for preventing or treating the orange-peel appearance of the skin, which includes topically applying a composition containing at least one selected from the group including ergothioneine, derivatives thereof, and mixtures thereof to the skin of a person in need thereof.

### DESCRIPTION OF THE PREFERRED EMBODIMENTS

Various other objects, features and attendant advantages of the present invention will be more fully appreciated as the same becomes better understood from the following detailed description of the preferred embodiments of the invention.

The present inventors have discovered, surprisingly and unexpectedly, that ergothioneine and its derivatives have the property of reducing or even inhibiting the phenomenon of glycation of proteins, age-related loss of tonicity of the skin, and “orange-peel” appearance of the skin. In addition, in accordance with the present invention, the anti-glycant effect of ergothioneine finds an advantageous application in facial slimming products intended to redesign the contours of the face which have been deformed by age.

Ergothioneine has already been disclosed as a melanogenesis inhibitor (Motohashi, N. et al., Chem. Pharm. Bull., 39(1), 142-5 (1991) and JP-63 008 335) and as a free-radical scavenger (Whiteman M. et al., FEBS Lett., 414(3), 497-500 (1997), the entire contents of each of which are hereby incorporated by reference). However, it is believed that it has never been suggested that ergothioneine might have glycation-inhibiting properties.

The skin proteins are preferably the proteins of the dermis. The compounds according to the invention may thus be used to reduce the glycation of collagen. This term means any type of collagen present in the skin.

According to the invention, ergothioneine and/or its derivatives may thus be used:

to prevent or treat the signs of glycation-related ageing of the skin, in particular to prevent or treat age-related loss of tonicity of the skin; and/or

to slim and/or refine the silhouette and/or the contours of the face; and/or

to prevent or treat the orange-peel appearance of the skin.

For the purposes of the present invention, ergothioneine and/or its derivatives preferably correspond to formula (I):

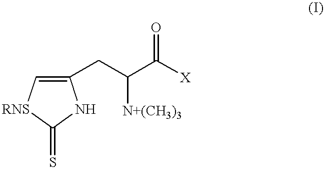

in which:

X represents a group —O—R′ or —NR′R″;

R, R′ and R″ represent, simultaneously or independently of each other, a hydrogen atom, an alkyl group, an alkenyl group or an acyl group which is C₁-C₁₈, these groups being linear or branched and optionally substituted with:

(a) an optionally esterified hydroxyl group, and/or

(b) a halogen atom, and/or

(c) a carboxylic group, and/or

(d) an amine group.

According to one preferred embodiment of the invention, R denotes a hydrogen atom or an ethyl or acetyl group and X denotes NH₂. In particular, it is preferred to use ergothioneine, preferably L-(+)-ergothioneine.

Preferably, R, R′ and R″ represent, simultaneously or independently of each other, a hydrogen atom, a linear or branched, substituted or unsubstituted C₁-C₁₈ alkyl group, C₂-C₁₈ alkenyl group or C₁-C₁₈ acyl group. Each R, R′ and R″ may be independently substituted with one or more of the following:

(a) an optionally esterified hydroxyl group;

(b) a halogen atom;

(c) a carboxylic group;

(d) an amine group;

and combinations thereof.

Among the R, R′ and R″ groups, carbon numbers of 1, 2, 3, 4, 5, 6, 7, 8, 9, 10, 11, 12, 13, 14, 15, 16, 17 and 18 are appropriately included. Among the possible halogen atom substituents, chloride, bromide, fluoride and iodide are included.

The composition also preferably includes a suitable counterion for balancing the positive charge on the above-identified compound of formula (I). Preferably, the counterion is a halide such as chloride, bromide, fluoride, or iodide; any of phosphate, sulfate, sulfonates, methane sulfonate, citrate, acetate, lactate or glycolate; or any other physiologically acceptable anion.

The composition according to the invention is preferably intended for cosmetic or dermatological use and advantageously cosmetic use. It is preferably intended for topical application to the skin and thus generally contains a physiologically acceptable medium, that is to say a medium which is compatible with the skin.

Preferably, the ergothioneine and/or its derivative is present or is applied in an effective amount to achieve the desired object. Preferably, the effective amount is the amount effective for reducing or inhibiting the glycation of skin proteins, for reducing the glycation of collagen, for preventing or treating the signs of glycation-related ageing of the skin, for preventing or treating age-related loss of tonicity of the skin, for slimming or refining the silhouette or contours of the face, or for preventing or treating the orange-peel appearance of the skin.

The ergothioneine and/or its derivative is preferably present in an amount ranging from 0.001 to 10% relative to the total weight of the composition and better still in an amount ranging from 0.005 to 5% relative to the total weight of the composition. These ranges particularly include 0.002, 0.007, 0.01, 0.05, 0.1, 0.5, 1, 2, 3, 4, 6, 7, 8, and 9%.

This composition may be in any pharmaceutical form normally used in cosmetics and dermatology, and may especially be in the form of an optionally gelled aqueous solution, an optionally two-phase dispersion of the lotion type, an emulsion obtained by dispersing a fatty phase in an aqueous phase (O/W emulsion) or vice versa (W/O emulsion) or a triple emulsion (W/O/W or O/W/O emulsion) or a vesicular dispersion of ionic and/or nonionic type. These compositions are prepared according to the usual methods.

This composition may be more or less fluid and may have the appearance of a white or coloured cream, an ointment, a milk, a lotion, a serum, a paste or a mousse. It may optionally be applied to the skin in the form of an aerosol. It may also be in solid form, in particular in the form of a stick. It may be used as a skincare product and/or as a make-up product for the skin.

In a known manner, the composition of the invention may also contain adjuvants that are common in cosmetics, such as hydrophilic or lipophilic gelling agents, hydrophilic or lipophilic active agents, preserving agents, antioxidants, solvents, fragrances, fillers, screening agents, pigments, odour absorbers, dyestuffs, and mixtures thereof. The amounts of these various adjuvants are those used conventionally in the field under consideration, and, for example, from 0.01 to 20% relative to the total weight of the composition. This range particularly includes 0.05, 0.1, 0.5, 1, 2, 3, 5, 7, 9, 11, 13, 15, 17, and 19%. Depending on their nature, these adjuvants may be introduced into the fatty phase, into the aqueous phase, into lipid vesicles and/or into nanoparticles. In any case, these adjuvants, and the proportions thereof, will be chosen so as not to harm the desired properties of the combination of active agents according to the invention.

When the composition of the invention is an emulsion, the proportion of the fatty phase may range from 5% to 80% by weight and preferably from 5% to 50% by weight relative to the total weight of the composition. These ranges particularly include 7, 10, 15, 20, 25, 30, 35, 40, 45, 55, 60, 65, 70 and 75%. The oils, emulsifiers and co-emulsifiers used in the composition in emulsion form are chosen from those used conventionally in the field under consideration. The emulsifier and the co-emulsifier are present in the composition in a proportion ranging from 0.3% to 30% by weight and preferably from 0.5% to 20% by weight relative to the total weight of the composition. These ranges particularly include 0.7, 0.9, 1, 2, 5, 7, 11, 15, 17, 22, 25 and 27%. Mixtures are possible.

As oils which may be used in the invention, mention may be made of mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (camauba wax or ozokerite) may also be used as fatty substances. Mixtures are possible.

As emulsifiers and co-emulsifiers which may be used in the invention, mention may be made, for example, of fatty acid esters of polyethylene glycol, such as PEG-20 stearate, and fatty acid esters of glycerol, such as glyceryl stearate. Mixtures are possible.

Hydrophilic gelling agents which may be mentioned in particular are carboxyvinyl polymers (carbomer), acrylic copolymers such as acrylate/alkyl acrylate copolymers, polyacrylamides, polysaccharides, natural gums and clays, and lipophilic gelling agents which may be mentioned are modified clays, for instance bentones, metal salts of fatty acids, hydrophobic silica and polyethylenes. Mixtures are possible.

Preferably, and particularly to prevent or treat the loss of tonicity of the skin and/or to refine the contours of the face, the ergothioneine may be combined with other anti-ageing active agents, in particular anti-wrinkle, depigmenting, firming, draining, tensioning, free-radical-scavenging and/or immunoprotective active agents and/or with agents for stimulating cell metabolism (in particular protein synthesis) and/or cell proliferation.

Preferably, and particularly to refine the silhouette and/or to prevent or treat the orange-peel appearance of the skin, the ergothioneine may be combined with firming, draining, astringent, lipolytic, tensioning and/or desquamating active agents.

Preferably, and particularly in the event of incompatibility, at least some of the active agents mentioned above may be incorporated into spherules, in particular ionic or nonionic vesicles and/or nanoparticles (nanocapsules and/or nanospheres), such that the active agents that are mutually incompatible are isolated from each other in the composition.

The invention relates to the use of ergothioneine and/or its derivatives in a cosmetic composition, and/or for the manufacture of a dermatological composition, for preventing or treating the loss of tonicity of the skin, in particular due to age, and also to its use in a cosmetic composition for preventing or treating the orange-peel appearance of the skin. This composition may in particular be for anti-ageing or slimming purposes. Specifically, it has been discovered that ergothioneine has the property of reducing the glycation of proteins, in particular the proteins of the dermis such as collagen, which is responsible for the rigidification of tissues.

### EXAMPLES

Having generally described this invention, a further understanding can be obtained by reference to certain specific examples which are provided herein for purposes of illustration only and are not intended to be limiting unless otherwise specified.

### Example 1

### Study of the Effect of Ergothioneine on Glycation

The formation of the glycation products is observed by placing together a bovine serum albumin (BSA) and D-ribose in the absence or presence of ergothioneine. The inhibition of glycation is measured by the variation in the fluorescence emitted.

Specifically, a solution of bovine serum albumin at a concentration of 1 mg/ml dissolved in phosphate-buffered saline (PBS) is incubated at 37° C. for 14 days with D-ribose at a concentration of 10 mM, in the presence or absence of ergothioneine at concentrations of 500 μM, 1 mM or 5 mM.

The glycation is evaluated by measuring the fluorescence of the AGES at λem.=440 nm emitted by each sample after excitation at λex.=370 nm. The inhibition of glycation is visualized by the decrease in fluorescence compared with the sample treated with the sugar alone.

The results are collated in Table 1 below and expressed as a percentage inhibition of fluorescence relative to the glycated control:

TABLE 1
Inhibition of glycation

| | λex 370/λem 440 nm |
|---|---|
| 500 μM ergothioneine | 19.0% |
| 1 mM ergothioneine | 28.6% |
| 5 mM ergothioneine | 42.9% |

Ergothioneine thus appears to have advantageous anti-glycation activity, which is dependent on the concentration of active agent.

### Example 2

### Cosmetic Compositions

The amounts of the constituents of the compositions given by way of illustration below are indicated as percentages by weight. These compositions are prepared according to the usual techniques that are well known to those skilled in the art.

Example 2-1: Anti-ageing cream

| | | |
|---|---|---|
| Ergothioneine | | 1% |
| Sorbitan tristearate | | 0.9% |
| Polyethylene glycol stearate (40 EO) | | 2% |
| Cetyl alcohol | | 4% |
| Glyceryl stearate | | 3% |
| Stearic acid | | 1.2% |
| Petroleum jelly | | 4% |
| Hydrogenated polyisobutene | | 8.5% |
| Myristyl myristate | | 2% |
| Cyclopentasiloxane | | 5% |
| Glycerol | | 3% |
| Preserving agents | | 0.3% |
| Sodium hydroxide | | 0.05% |
| Water | qs | 100% |

Example 2-2: Slimming composition

| | | |
|---|---|---|
| Ergothioneine | | 1% |
| PVM/MA decadiene copolymer | | 0.45% |
| Cyclohexasiloxane | | 7% |
| Carbomer | | 0.3% |
| Triethanolamine | | 0.5% |
| Propylene glycol | | 3% |
| Caffeine | | 2% |
| Gelling agent (Sepigel 305 from the | | 0.25% |
| company SEPPIC) | | |
| Water | qs | 100% |

Example 2-3: Anti-“orange-peel skin” cream

| | | |
|---|---|---|
| Ergothioneine | | 1% |
| Sodium dimethicone copolyol acetyl methyltaurate | | 5.4% |
| (Pecosil DCT from the company PHOENIX) | | |
| Mineral oil | | 19% |
| Glycerol | | 5% |
| Preserving agents | | 0.25% |
| Caffeine | | 1% |
| Xanthan gum | | 0.25% |
| Ammonium salt of | | 1.2% |
| acrylamidomethylpropane-sulphonic acid | | |
| Mixture of cyclopentasiloxane and dimethiconol | | 5% |
| Extract of Ginkgo biloba in propylene glycol | | 5% |
| Water | qs | 100% |

Obviously, numerous modifications and variations of the present invention are possible in light of the above teachings. It is therefore to be understood that within the scope of the appended claims, the invention may be practiced otherwise than as specifically described herein.

This application is based on French patent application 0008158, filed Jun. 26, 2000, the entire contents of which are hereby incorporated by reference, the same as if set forth at length.

1. A method for reducing or inhibiting the glycation of skin proteins, comprising topically applying a composition comprising at least one selected from the group consisting of ergothioneine, derivatives thereof, and mixtures thereof to the skin of a person in need thereof.

2. The method according to claim 1, wherein said skin proteins are proteins of the dermis.

3. The method according to claim 1, wherein the ergothioneine and/or the derivatives correspond to formula (I): 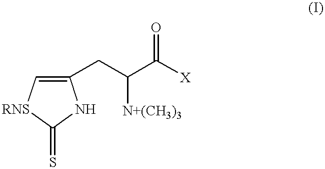
wherein:
X represents a group —O—R′ or —NR′R″; and
R, R′ and R″ represent, simultaneously or independently of each other, a hydrogen atom, a linear or branched, substituted or unsubstituted C₁-C₁₈ alkyl group, C₂-C₁₈ alkenyl group or C₁-C₁₈ acyl group, each R, R′ and R″ being optionally independently substituted with one or more of the following:
(a) an optionally esterified hydroxyl group;
(b) a halogen atom;
(c) a carboxylic group;
(d) an amine group;
and combinations thereof.

4. The method according to claim 3, wherein R denotes a hydrogen atom or an ethyl or acetyl group and X denotes NH₂.

5. The method according to claim 3, wherein the compound of formula (1) is ergothioneine.

6. The method according to claim 3, wherein the compound of formula (1) is L-(+)-ergothioneine.

7. The method according to claim 1, wherein said composition further comprises a physiologically acceptable medium.

8. The method according to claim 1, wherein the ergothioneine and/or its derivative is present in an amount ranging from 0.001 to 10% relative to the total weight of the composition.

9. The method according to claim 1, wherein the ergothioneine and/or its derivative is present in an amount ranging from 0.005 to 5% relative to the total weight of the composition.

10. A method for making a composition for reducing the glycation of skin proteins, comprising contacting at least one selected from the group consisting of ergothioneine, derivatives thereof, and mixtures thereof with a physiologically acceptable medium.

11. A method for reducing the glycation of collagen, comprising topically applying a composition comprising at least one selected from the group consisting of ergothioneine, derivatives thereof, and mixtures thereof to the skin of a person in need thereof.

12. A method for preventing or treating the signs of glycation-related ageing of the skin, comprising topically applying a composition comprising at least one selected from the group consisting of ergothioneine, derivatives thereof, and mixtures thereof to the skin of a person in need thereof.

13. A method for preventing or treating age-related loss of tonicity of the skin, comprising topically applying a composition comprising at least one selected from the group consisting of ergothioneine, derivatives thereof, and mixtures thereof to the skin of a person in need thereof.

14. A method for slimming or refining the silhouette or contours of the face, comprising topically applying a composition comprising at least one selected from the group consisting of ergothioneine, derivatives thereof, and mixtures thereof to the skin of a person in need thereof.

15. A method for preventing or treating the orange-peel appearance of the skin, comprising topically applying a composition comprising at least one selected from the group consisting of ergothioneine, derivatives thereof, and mixtures thereof to the skin of a person in need thereof.
